SEQUENCE LISTING (108 sequences)

SEQ ID NO: 1 - DNA, 20 bp, Artificial Sequence
  Synthesized sequence
taatacgactcactataggg

SEQ ID NO: 2 - DNA, 18 bp, Artificial Sequence
  Synthetic Sequence
tttaaccgcgaattccag

SEQ ID NO: 3 - DNA, 34 bp, Artificial Sequence
  Synthetic Sequence
cuuuuucccccucgcggctgtaggcaccatcaat

SEQ ID NO: 4 - DNA, 34 bp, Artificial Sequence
  Synthetic Sequence
gaaaaagggggagcgccgacatccgtggtagtta

SEQ ID NO: 5 - DNA, 22 bp, Artificial Sequence
  Synthetic Sequence
cgaattacgacttgcgtataga

SEQ ID NO: 6 - DNA, 22 bp, Artificial Sequence
  Synthetic Sequence
cgaccgataattatctatccgc

SEQ ID NO: 7 - DNA, 22 bp, Artificial Sequence
  Synthetic Sequence
cgttaaacgattgattacgtcg

SEQ ID NO: 8 - DNA, 22 bp, Artificial Sequence
  Synthetic Sequence
ccgacgtataacgcatactatc

SEQ ID NO: 9 - DNA, 22 bp, Artificial Sequence
  Synthetic sequence
cgtaatcgctaacttacgagtc

SEQ ID NO: 10 - DNA, 22 bp, Artificial Sequence
  Synthetic sequence
cggattacgtatatcgttacgc

SEQ ID NO: 11 - DNA, 22 bp, Artificial Sequence
  Synthetic Sequence
tacgacgctaacttatccgatc

SEQ ID NO: 12 - DNA, 22 bp, Artificial Sequence
  Synthetic Sequence
cgaattacgaccggagctataa

SEQ ID NO: 13 - DNA, 22 bp, Artificial Sequence
  Synthetic sequence
acggtcgataactcatatcgtg

SEQ ID NO: 14 - DNA, 22 bp, Artificial Sequence
  Synthetic sequence
cgaaacgttatcgcgtactaag

SEQ ID NO: 15 - DNA, 22 bp, Artificial Sequence
  Synthetic sequence
tatcgctaccgtagttaatcgc

SEQ ID NO: 16 - DNA, 22 bp, Artificial Sequence
  Synthetic sequence
taacgtacgaaccgatgtagtg

SEQ ID NO: 17 - DNA, 22 bp, Artificial Sequence
  Synthetic sequence
cgaatttagcgcgactcttatg

SEQ ID NO: 18 - DNA, 22 bp, Artificial Sequence
  Synthetic sequence
actaatcgcgaatcgggttatc

SEQ ID NO: 19 - DNA, 22 bp, Artificial Sequence
  Synthetic sequence
tcgctaaaccgttagatcgaac

SEQ ID NO: 20 - DNA, 22 bp, Artificial Sequence
  Synthetic sequence
taacgtacgaaccgaacttacg

SEQ ID NO: 21 - DNA, 22 bp, Artificial Sequence
  Synthetic sequence
cgactaaatcgcgacccataat

SEQ ID NO: 22 - DNA, 22 bp, Artificial Sequence
  Synthetic sequence
tagtcgaacgatcgttacaacg

SEQ ID NO: 23 - DNA, 22 bp, Artificial Sequence
  Synthetic sequence
cgctacgtaacgccaatttaac

SEQ ID NO: 24 - DNA, 22 bp, Artificial Sequence
  Synthetic sequence
tcgactaaacgtgccgaattag

SEQ ID NO: 25 - DNA, 22 bp, Artificial Sequence
  Synthetic sequence
gcgattaacgagacgacgtatt

SEQ ID NO: 26 - DNA, 22 bp, Artificial Sequence
  Synthetic sequence
acttatcgacgagtcagcgtat

SEQ ID NO: 27 - DNA, 22 bp, Artificial Sequence
  Synthetic sequence
cgtacgaatctacaagagacgc

SEQ ID NO: 28 - DNA, 22 bp, Artificial Sequence
  Synthetic sequence
tcgactaaacgacccaatcgta

SEQ ID NO: 29 - DNA, 22 bp, Artificial Sequence
  Synthetic sequence
tatcgctaccgcgtagattgat

SEQ ID NO: 30 - DNA, 22 bp, Artificial Sequence
  Synthetic sequence
cgatcaacgtatatgacgacgg

SEQ ID NO: 31 - DNA, 22 bp, Artificial Sequence
  Synthetic sequence
cgatcgacttacgccgtaattt

SEQ ID NO: 32 - DNA, 22 bp, Artificial Sequence
  Synthetic sequence
tatgcgtaacgtaccgctctta

SEQ ID NO: 33 - DNA, 22 bp, Artificial Sequence
  Synthetic sequence
atcgacgcttatcataccggtt

SEQ ID NO: 34 - DNA, 22 bp, Artificial Sequence
  Synthetic sequence
ttagcgtatcgacccaatcgtt

SEQ ID NO: 35 - DNA, 22 bp, Artificial Sequence
  Synthetic sequence
atcgtacgatacgcctattcgg

SEQ ID NO: 36 - DNA, 22 bp, Artificial Sequence
  Synthetic sequence
ctatccgaacgataaggccgaa

SEQ ID NO: 37 - DNA, 22 bp, Artificial Sequence
  Synthetic sequence
cgcgactaaatgttcgagatcg

SEQ ID NO: 38 - DNA, 22 bp, Artificial Sequence
  Synthetic sequence
cgaattacgacggtcattgagc

SEQ ID NO: 39 - DNA, 22 bp, Artificial Sequence
  Synthetic sequence
atcgacgctagcggtgaattta

SEQ ID NO: 40 - DNA, 22 bp, Artificial Sequence
  Synthetic sequence
taccgtaacgcgatcctttacc

SEQ ID NO: 41 - DNA, 22 bp, Artificial Sequence
  Synthetic sequence
tcgatttagcgacgtcttaccc

SEQ ID NO: 42 - DNA, 22 bp, Artificial Sequence
  Synthetic sequence
taacgtacgacgcaagggatac

SEQ ID NO: 43 - DNA, 22 bp, Artificial Sequence
  Synthetic sequence
taatcgtacgggttatggcgtc

SEQ ID NO: 44 - DNA, 22 bp, Artificial Sequence
  Synthetic sequence
atcgtacgatactcgatccgga

SEQ ID NO: 45 - DNA, 22 bp, Artificial Sequence
  Synthetic sequence
tacgatcggaacggaaataccg

SEQ ID NO: 46 - DNA, 22 bp, Artificial Sequence
  Synthetic sequence
tacgacgctaatacctcggcta

SEQ ID NO: 47 - DNA, 22 bp, Artificial Sequence
  Synthetic sequence
tatcgctaccgtacgctcagta

SEQ ID NO: 48 - DNA, 22 bp, Artificial Sequence
  Synthetic sequence
atcgaaccgtaataacgcgaca

SEQ ID NO: 49 - DNA, 22 bp, Artificial Sequence
  Synthetic sequence
atcgatcgttatacgtagcgcc

SEQ ID NO: 50 - DNA, 22 bp, Artificial Sequence
  Synthetic sequence
gcgaaccgatagtacgtccaat

SEQ ID NO: 51 - DNA, 22 bp, Artificial Sequence
  Synthetic sequence
tatgcgtaacgtaggcgaagac

SEQ ID NO: 52 - DNA, 22 bp, Artificial Sequence
  Synthetic sequence
acggtcgataacgcgaaattct

SEQ ID NO: 53 - DNA, 22 bp, Artificial Sequence
  Synthetic sequence
atcgatcgttaaacgacgcact

SEQ ID NO: 54 - DNA, 22 bp, Artificial Sequence
  Synthetic sequence
cgatcgacttacggcgaagatt

SEQ ID NO: 55 - DNA, 22 bp, Artificial Sequence
  Synthetic sequence
cgaattacgacgtgatagccgt

SEQ ID NO: 56 - DNA, 22 bp, Artificial Sequence
  Synthetic sequence
taatcgtacggcgccaatttga

SEQ ID NO: 57 - DNA, 22 bp, Artificial Sequence
  Synthetic sequence
agcgtaaaccgttcgagaagtc

SEQ ID NO: 58 - DNA, 22 bp, Artificial Sequence
  Synthetic sequence
ctacggaacgacgatcgagttc

SEQ ID NO: 59 - DNA, 22 bp, Artificial Sequence
  Synthetic sequence
cgttaaacgatggcgaacaacc

SEQ ID NO: 60 - DNA, 22 bp, Artificial Sequence
  Synthetic sequence
atacgacgttaacgtaacggcc

SEQ ID NO: 61 - DNA, 22 bp, Artificial Sequence
  Synthetic sequence
ctaatcgaacgggacgagaacg

SEQ ID NO: 62 - DNA, 22 bp, Artificial Sequence
  Synthetic sequence
actaatcgcgatggtccgtagt

SEQ ID NO: 63 - DNA, 22 bp, Artificial Sequence
  Synthetic sequence
tacgtaacggcgcactataggt

SEQ ID NO: 64 - DNA, 22 bp, Artificial Sequence
  Synthetic sequence
taatcgtacgggacgtgatcgt

SEQ ID NO: 65 - DNA, 22 bp, Artificial Sequence
  Synthetic sequence
tacgatcggaacgggtaacctc

SEQ ID NO: 66 - DNA, 22 bp, Artificial Sequence
  Synthetic sequence
taatcgtacggttcgcgagagt

SEQ ID NO: 67 - DNA, 22 bp, Artificial Sequence
  Synthetic sequence
cgtacaatcgacgcgatctacg

SEQ ID NO: 68 - DNA, 22 bp, Artificial Sequence
  Synthetic sequence
tcgacgtaacgacgctagaaca

SEQ ID NO: 69 - DNA, 22 bp, Artificial Sequence
  Synthetic sequence
taacgtacgacaggcgatcgaa

SEQ ID NO: 70 - DNA, 22 bp, Artificial Sequence
  Synthetic sequence
gaacgtacgtaatatcgcccgc

SEQ ID NO: 71 - DNA, 22 bp, Artificial Sequence
  Synthetic sequence
taccgtaatcgcgcaataggct

SEQ ID NO: 72 - DNA, 22 bp, Artificial Sequence
  Synthetic sequence
cgacgttatactcgccgattgc

SEQ ID NO: 73 - DNA, 22 bp, Artificial Sequence
  Synthetic sequence
cgcgactaaatcgggtaccagt

SEQ ID NO: 74 - DNA, 22 bp, Artificial Sequence
  Synthetic sequence
cgaattacgacgcgcgataagg

SEQ ID NO: 75 - DNA, 22 bp, Artificial Sequence
  Synthetic sequence
actaatcgcgatcctcgttcgg

SEQ ID NO: 76 - DNA, 22 bp, Artificial Sequence
  Synthetic sequence
gcgctacgtaacgtacattcgc

SEQ ID NO: 77 - DNA, 22 bp, Artificial Sequence
  Synthetic sequence
ctatccgaacgcgacctgtcag

SEQ ID NO: 78 - DNA, 22 bp, Artificial Sequence
  Synthetic sequence
tatgcgtaacgccctaacagcg

SEQ ID NO: 79 - DNA, 22 bp, Artificial Sequence
  Synthetic sequence
cgctaatcgacatccgcacagt

SEQ ID NO: 80 - DNA, 22 bp, Artificial Sequence
  Synthetic sequence
ctaccgtaacgcgacacatccc

SEQ ID NO: 81 - DNA, 22 bp, Artificial Sequence
  Synthetic sequence
tatacgtccgaggcgctacgtt

SEQ ID NO: 82 - DNA, 22 bp, Artificial Sequence
  Synthetic sequence
taacgctacgcatggttcgtcg

SEQ ID NO: 83 - DNA, 22 bp, Artificial Sequence
  Synthetic sequence
tcgactaagcgcgccgatttac

SEQ ID NO: 84 - DNA, 22 bp, Artificial Sequence
  Synthetic sequence
cgatcgacttaccgcccgtaga

SEQ ID NO: 85 - DNA, 22 bp, Artificial Sequence
  Synthetic sequence
tctaaccgacgaacgatcccgg

SEQ ID NO: 86 - DNA, 22 bp, Artificial Sequence
  Synthetic sequence
tcgatttagcgccgtcgtctga

SEQ ID NO: 87 - DNA, 22 bp, Artificial Sequence
  Synthetic sequence
cgtaaccgactacctttgcgcg

SEQ ID NO: 88 - DNA, 22 bp, Artificial Sequence
  Synthetic sequence
cgctaatcgacccgccgatact

SEQ ID NO: 89 - DNA, 22 bp, Artificial Sequence
  Synthetic sequence
ccgactaacgggctaagtgcga

SEQ ID NO: 90 - DNA, 22 bp, Artificial Sequence
  Synthetic sequence
tcgtacgttaaggcccggaacg

SEQ ID NO: 91 - DNA, 22 bp, Artificial Sequence
  Synthetic sequence
ctacggaacgaacacgccgagt

SEQ ID NO: 92 - DNA, 22 bp, Artificial Sequence
  Synthetic sequence
gcgaaccgatagaaccgcgagt

SEQ ID NO: 93 - DNA, 22 bp, Artificial Sequence
  Synthetic sequence
ctaccgtaacggacacctcgcg

SEQ ID NO: 94 - DNA, 22 bp, Artificial Sequence
  Synthetic sequence
agcgtaaaccgtctgcgactcg

SEQ ID NO: 95 - DNA, 22 bp, Artificial Sequence
  Synthetic sequence
gacgtaaccgaaccgatgcccc

SEQ ID NO: 96 - DNA, 22 bp, Artificial Sequence
  Synthetic sequence
tacgcctaacgcacgctcaagc

SEQ ID NO: 97 - DNA, 22 bp, Artificial Sequence
  Synthetic sequence
acgtctaaacgccgtgcgcatt

SEQ ID NO: 98 - DNA, 22 bp, Artificial Sequence
  Synthetic sequence
cgactaacgctcccgctcgaga

SEQ ID NO: 99 - DNA, 22 bp, Artificial Sequence
  Synthetic sequence
cgtaacgattatgcgggccgcc

SEQ ID NO: 100 - DNA, 22 bp, Artificial Sequence
  Synthetic sequence
aacgtaccggacgcatcgcagt

SEQ ID NO: 101 - DNA, 22 bp, Artificial Sequence
  Synthetic sequence
tcgacgtaacgcgcaggcctac

SEQ ID NO: 102 - DNA, 22 bp, Artificial Sequence
  Synthetic sequence
cgcctaacgtaccccgcggagt

SEQ ID NO: 103 - DNA, 22 bp, Artificial Sequence
  Synthetic sequence
ctacggaacgaggccccgcgca

SEQ ID NO: 104 - DNA, 18 bp, Artificial Sequence
  Synthetic sequence
ctggaattcgcggttaaa

SEQ ID NO: 105 - RNA, 53 nt, Artificial Sequence
  Synthetic sequence
agcuuaaaaaaaaaaaaaaaaaaaaaaaaaaaaaauuuaaccgcgaauuccag

SEQ ID NO: 106 - DNA, 53 bp, Artificial Sequence
  Synthetic sequence
agcttaaaaaaaaaaaaaaaaaaaaaaaaaaaaaatttaaccgcgaattccag

SEQ ID NO: 107 - RNA, 68 nt, Artificial Sequence
  Synthetic sequence
agcuuaaaaaaaaaaaaaaaaaaaaaaaaaaaaaaaaaaaaaaaaaaaaauuuaaccgcgaauuccag

SEQ ID NO: 108 - DNA, 68 bp, Artificial Sequence
  Synthetic sequence
agcttaaaaaaaaaaaaaaaaaaaaaaaaaaaaaaaaaaaaaaaaaaaaatttaaccgcgaattccag

(19) United States
(12) Patent Application Publication
(10) Pub. No.: US 2011/0269137 A1
(43) Pub. Date: Nov. 3, 2011

(54) RAPID AND EFFICIENT ASSAY TO ASSESS THE SEQUENCE AND SIZE OF 3' ENDS OF POLYNUCLEOTIDES

(76) Inventors: Lance P. Ford, Austin, TX (US); Masoud Toloue, Austin, TX (US); Marianna Goldrick, (US); Jeffrey Wilusz, Fort Collins, CO (US)

(21) Appl. No.: 13/089,062

(22) Filed: Apr. 18, 2011

Related U.S. Application Data
(60) Provisional application No. 61/325,144, filed on Apr. 16, 2010.

(51) Int. Cl. C12Q 1/68

(52) U.S. Cl. 435/6.12; 435/6.1

(57) ABSTRACT

Described herein is an efficient, highly reproducible approach to assess poly(A) tail length on a mRNA specific basis. The embodiments herein have led to the development of a versatile, easy to use kit for biomedical researchers to address the impact of changes in poly(A) tail length in the post-transcriptional regulation of gene expression.

### PRIORITY CLAIM

This application claims the benefit of U.S. Provisional Application No. 61/325,144 filed on Apr. 16, 2010.

### STATEMENT REGARDING FEDERALLY SPONSORED RESEARCH OR DEVELOPMENT

This invention was made with government support from the National Institute of General Medical Sciences (NIH-NIGMS), Grant number 1R43GM084602-01. The U.S. Government has certain rights to this invention.

### BACKGROUND OF THE INVENTION

1. Field of the Invention

This invention generally relates to a rapid, efficient and highly sensitive means to assess the sequence of polynucleotides. More particularly, the invention relates to means of assessing the size of any 3′ end on a polynucleotide, and in particular 3′ poly(A) tail lengths on mRNAs, from any organism or biological system.

2. Description of the Relevant Art

One of the major underlying factors for the significant broad impact of molecular biology on biomedical research is the generation of effective and highly reproducible assay kits to measure fundamental properties of gene expression. One area of molecular biology that is relatively understudied is the role of poly(A) tail length in the regulation of transcript fate and gene expression due to the absence of a highly reproducible means for the average biomedical research lab to assess this property of cellular mRNAs. Current assays have enjoyed varied success among laboratories and many standard molecular biology researchers simply avoid assessing poly(A) tail length due to the lack of a readily available approach.

The vast majority of eukaryotic mRNAs contain a poly(A) tail at their 3′ end as a result of a cleavage/polyadenylation process that occurs co-transcriptionally in the nucleus. The poly(A) tail plays an important role in a variety of cell processes, including export, translation, and mRNA stability. Interestingly, the length or the site of addition of the poly(A) tail is not static, but rather varies due to synthetic, degradative or remodeling processes. This suggests that the poly(A) tail may serve as an adjustable regulator of gene expression that can serve to coordinate expression among functional classes of transcripts as well as a means of quickly adjusting cellular gene expression to address environmental changes. Recent global analyses have highlighted the importance of the changes in poly(A) tail length related to gene expression. Global studies in S. cerevisiae and fission yeast, for example, have demonstrated an extensive correlation between tail length and mRNA functions. The poly(A) tail length of sets of genes involved in functionally-related mRNAs appear to be co-regulated, suggesting that the adjustment of poly(A) tail length may be a way of fine-tuning gene expression for members of post-transcriptionally regulated ‘operons’.

There are several lines of evidence that suggest that poly(A) tail length represents a ‘hidden’ form of gene expression:
Changes in poly(A) tail length are a major regulator of gene expression early in development. Through the concerted action of cytoplasmic polyadenylation and deadenylation, the poly(A) tail length of key developmental mRNAs is altered to either activate or repress expression to coordinate cell development/differentiation. Although there is some evidence, most notably in neurons, that changes in poly(A) tail length occurs in somatic cells, the extent of this type of regulation clearly needs to be fully explored. An easy method to assess changes in poly(A) tail length over time in select transcripts among a population of mRNAs, therefore, should yield new insights into the underlying kinetics of deadenylation rates in vivo as well as help address the identity of the responsible deadenylase (by allowing fine assessment of poly(A) length changes in knock downs).
There is a strong association of poly(A) tail length and translation efficiency. The extent that poly(A) tail remodeling serves to fine tune gene expression at the translational level remains to be determined for most genes.
Deadenylation, the first step in the major pathway of mRNA decay, appears to occur through a series of staged steps of poly(A) tail removal, perhaps involving different deadenylase enzymes. Understanding the dynamics of poly(A) tail shortening, the often rate-limiting step in mRNA turnover, will clearly benefit from effective assays to measure poly(A) tail lengths.
Polyadenylation limiting elements, which limit the overall length of the poly(A) tail to ˜25 bases, have been described for several genes. Deciphering the full impact of these elements on gene expression will greatly benefit from a readily available assay to size poly(A) tails.
A sizeable number of non-classical poly(A) and poly(U) polymerases have been detected in the nucleus and cytoplasm of eukaryotic cells. These have been hypothesized to play a number of roles in the cell. For example, the poly(A) polymerase of the TRAMP complex serves to add a short tail onto the end of mis-folded structural RNAs in yeast to provide a landing pad for the exosome complex and promote their decay. Therefore poly(A) tails, and homopolymeric tails in general, are by no means limited to mRNAs and appear to play a significant role in the cell—including the quality control of gene expression. Deciphering the role of these nuclear and cytoplasmic enzymes in the cell will require a readily available assay to assess the product of their activity—namely 3′ end extensions of homopolymer tracts on RNA targets. Assays to assess poly(A) tail length, therefore, may have significant application even outside of the mRNA arena
Recent data suggest that changes in poly(A) tail length appears to be a key component of miRNA-mediated regulation of gene expression. This suggests that assessment of poly(A) tail status will be very important for understanding mechanisms and applications of this developing area of gene regulation.

The main challenge in assessing poly(A) tail lengths of most mRNAs is that the large size of the body of the message makes it difficult to detect changes in the tail due to the lack of resolving power of the gel system employed. To overcome this problem, there are two primary assays currently in use to assess the length of poly(A) tails on specific transcripts.

In the RNAse H cleavage/poly(A) tail assay, DNA oligos are designed to cleave the RNA body of the selected transcript in the 3′ UTR, usually within ˜100-200 bases of the start of the tail. RNAs are then separated on a denaturing 5% acrylamide gel, blotted, and probed to detect the fragment of interest. The size of the poly(A) tail is then estimated by comparison to size markers along with a sample that was treated with oligo dT/RNAse H to generate a fully deadenylated marker. While this assay is effective when successfully employed, it does have several drawbacks. These weaknesses include the length of time/manipulation required to cleave, blot and probe the assay, as well as the inconsistency in RNAse H-mediated cleavage by small DNA oligos due to interfering secondary structures. These weaknesses have limited the applications of this tool.

The second basic type of assay used to assess poly(A) tail length is one of three two-step PCR based approaches. The first step in the RACE-PAT method involves the priming of a RT step using an oligonucleotide containing oligo dT attached to a G-rich anchor sequence. This will generate an array of cDNAs since the oligo dT portion of the anchor primer can prime anywhere along the poly(A) tail. These cDNAs are then amplified using an upstream mRNA-specific primer in conjunction with an oligo that will prime from the anchor sequence (and may also contain a few nucleotides specific to the 3′ terminus of the polynucleotide). The results that are obtained when the PCR products are analyzed on a gel will represent an array of poly(A) tail sizes. This smear that is present on the gel, however, limits the sensitivity of this assay and does not make it suitable to detect small changes in poly(A) tail length over time. This represents a major limitation of this assay.

In the classic Ligation Mediated Poly(A) Tail (LM-PAT) assay, the poly(A) containing RNA is hybridized to an excess of kinased homopolymeric dT oligomers in the presence of T4 DNA ligase. The dT oligomers that hybridize to the tail get ligated together by the enzyme and therefore approximately serve as a DNA copy of the poly(A) tail. An oligo dT anchor primer is then hybridized to the remaining 3′ unpaired A residues and ligated to the 5′ end of the oligo dT chain. This anchor serves as the downstream PCR primer in the same manner described for the RACE-PAT assay above. While generally efficient and sensitive, this assay has received mixed reviews in labs due to the laddering that is often observed due to hybridization problems with dT oligomers used as well as other nuances with the hybridization step. Furthermore, since the dT oligos will hybridize randomly along the tail, one cannot be certain that the product truly represents a complete poly(A) tail.

A more recent addition to the PCR-based approaches for assessing poly(A) tail length is the ligation or enzymatic addition of non-adenosine residues directly to the end of the poly(A) tail to serve as a downstream primer for transcript-specific PCR amplification of the tail. The addition of non-A residues to the 3′ end was first approached by poly(G) tailing using yeast PAP. Although potentially powerful, this approach has not been widely used presumably due to inconsistencies with the poly(G) addition step.

Finally, in a variation of classic 3′ RACE, instead of using an oligo dT primer a separate anchor primer can be ligated to the 3′ end of the RNA. While this represents a potentially powerful approach that would overcome all of the limitations of described for the other assays, it does suffer from inefficient ligation issues which have limited its use.

In summary, the poly(A) length and poly(A) position site location for a given mRNA at certain times/conditions may represent a very important aspect of its regulation and biological impact. Numerous observations suggest that changes in poly(A) tail status in an RNA occur throughout its life span which can have very important implications for cell development, growth and differentiation. Molecular biologists clearly need the assays in order to accurately and efficiently assess this phenomenon.

### SUMMARY OF THE INVENTION

In one embodiment, a method of determining the sequence of an RNA molecule, includes: ligating an oligonucleotide sequence to the 3′ end of the RNA molecule; reverse transcribing the ligated RNA molecule using a primer complementary to all or part of the ligated oligonucleotide sequence; amplifying the transcribed RNA to produce a mixture comprising multiple copies of the RNA molecule; and determining the sequence of the RNA molecules in the mixture. In some embodiments, the RNA molecule has a poly(A) tail, and wherein determining the sequence of the RNA molecule comprises determining the poly(A) tail length length of the RNA molecule.

In an embodiment, the oligonucleotide sequence comprises an ATP moiety coupled to the 5′ end, wherein the ATP moiety is removed during ligation of the oligonucleotide sequence to the 3′ ends of the RNA molecule. The ligation of the oligonucleotide sequence to the 3′ end of the RNA molecule may be performed in the absence of added ATP during ligation, when the oligonucleotide sequence comprises an ATP moiety. In some embodiments, the oligonucleotide sequence comprises a dideoxy nucleotide coupled to the 3′ end, wherein the ligated RNA molecule comprises the dideoxy nucleotide coupled to the 3′ end of the RNA molecule. The oligonucleotide sequence may be selected to have a melting temperature between about 40° C. to about 60° C. In some embodiments, ligating an oligonucleotide sequence to the 3′ end of the RNA molecule is performed in the presence of a bridging oligonucleotide.

In one embodiment, amplifying the transcribed RNA comprises using a Forward primer that hybridizes to a sequence near the 3′ end of the unique sequence of the RNA molecule and a Reverse primer that hybridizes to a sequence within the ligated oligonucleotide sequence.

In one embodiment, determining the sequence of the RNA molecules comprises mixing the multiple copies of the RNA molecule with a dye, wherein the signal strength of the bound dye corresponds to the length of the RNA molecule.

In an embodiment, determining the sequence of the RNA molecules in the mixture comprises determining 3′ end modifications of mRNAs from all biological organisms and systems. In an embodiment, determining the sequence of the RNA molecules in the mixture comprises assessing the 3′ end proximal sequences of RNA precursors of all types from all biological organisms and systems. In an embodiment, determining the sequence of the RNA molecules in the mixture comprises determining sites of transcription termination from all biological organisms and systems. In an embodiment, determining the sequence of the RNA molecules in the mixture comprises analyzing 3′ proximal sequences of RNA processing and decay intermediates from RNAs of all types from all biological organisms and systems. In an embodiment, determining the sequence of the RNA molecules in the mixture comprises determining and analyzing the 3′ proximal sequences of intergenic transcription products from all biological organisms and systems.

In an embodiment, the RNA molecule is an RNA molecule produce under disease state conditions. The method may also include determining the difference of poly adenylated tail length between RNA molecules in the disease state and RNA molecules in a non-disease state.

In an embodiment, a method for comparing 3′UTR/polyA sequences between RNA molecules, includes: ligating an oligonucleotide sequence to the 3′ ends of an RNA molecule; reverse transcribing the ligated RNA molecule using a primer complementary to all or part of the ligated sequence; amplifying a specific 3′UTR/polyA sequence using a Forward primer that hybridizes to a sequence near the 3′ end of the unique sequence of the RNA molecule and a Reverse primer that hybridizes to a sequence within the ligated oligonucleotide sequence, mixing a portion of the amplified product with T7 RNA polymerase, rNTPs, and an appropriate buffer containing one or more divalent cations to form an amplification mixture; incubating the amplification mixture to allow transcription of both strands of the PCR amplicon to produce multiple RNA copies of each strand of the amplicon, wherein the strands will hybridize to create double-stranded RNA with single-stranded tails complementary to the first 16 bases of the T7 promoter; adding a portion of the incubated amplification mixture to a matrix to form a matrix mixture, wherein the matrix is coupled to oligonucleotide having a sequence comprised of at least the first 16 bases of the T7 promoter; incubating the matrix mixture to allow hybridization of the single-strand tails complementary to the T7 promoter, generated during incubation of the amplification mixture, with the oligonucleotide on the matrix; recovering the matrix; resuspending the matrix with associated double-strand RNA in a suitable buffer containing suitable double-strand nucleic acid-specific dye; and assessing the extent of staining of the material treated with the double-strand nucleic acid-specific dye, wherein the extent of staining is positively correlated with the sequence of the RNA molecule.

In some embodiments, amplifying a specific 3′UTR/polyA sequence is carried out using PCR in a reaction volume of about 50 μL for about 25-30 cycles. Amplifying a specific 3′UTR/polyA sequence may be carried out using PCR, and wherein the primers contain a consensus T7 promoter sequence at their 5′ ends (T7 promoter: 5′-TAATACGACTCACTATAGGG). An example of an appropriate buffer is a buffer that includes one or more divalent cations contains Mg++ cations. In some embodiments, incubating the amplification mixture is performed for 1 hour at 37 C.

In one embodiment, the matrix comprises agarose beads. In another embodiment, the matrix comprises magnetic beads. In an embodiment, the matrix mixture is incubated for 15 minutes at room temperature. The matrix material may be recovered by centrifugation of the matrix mixture to separate the matrix from the fluid in the matrix mixture, and removing the supernatant fluid from the separated matrix. One example of a suitable double-strand nucleic acid-specific dye is SYBR Green.

In an embodiment, a kit for analyzing the sequence of an RNA molecule, includes one or more adenlyated linkers coupleable to an RNA molecule. A kit may also include: an RNA ligase and RNA ligase buffer solution; a reverse transcriptase enzyme, a reverse transcriptase buffer, and one or more RT primers specific for the reverse transcriptase enzyme; a DNA polymerase, a mixture of deoxyribonucleotide triphosphates, and one or more PCR primers specific for the DNA polymerase; one or more matrix materials, wherein the matrix material is coupled to oligonucleotide having a sequence comprised of at least the first 16 bases of an RNA polymerase promoter; and one or more RNA molecules having a known poly(A) tail length that are used for testing of the accuracy of a method performed using the component of the kit.

Dynamic changes in poly(A) tail (homopolymer tract of adenosine residues at the 3′ terminus) length and addition site location to substrates plays an important yet likely underappreciated role in cell biology due to the lack of a well-accepted, easy to use assay to determine poly(A) tail length. The method described herein is meant to overcome this obstacle by developing a sensitive, accurate and highly reproducible assay to measure poly(A) tail length of any RNA species. The herein described procedures should jump-start research in a variety of areas of science including mRNA stability, poly(A) tail remodeling, miRNA-mediated regulation, alternative polyadenylation and the developing field of the addition of homopolymeric tags to the ends of transcripts as markers for quality control.

FIG. 1 shows a diagram of the ALL-Tail kit process. It may also include permutations of this basic process and contain a bridging oligo for linker ligations or may exclude or have added steps not depicted. The main steps of this process are briefly outlined:

Step 1: Ligation of a Downstream Anchor Primer to mRNAs (or Any Relevant Polynucleotide Sequence of Interest)

To dramatically increase the efficiency and specificity of this step for our assay we have instituted the following two inventive steps:
a. The 3′ oligomer used in the ligation contains an ATP moiety at its 5′ end. This allows the ligation reaction to be done in the absence of added ATP in the buffer and ensures that the only portion ligatable in the reaction will be the RNA oligo to the 3′ end of endogenous cellular RNAs. This removes the problems of ligation of cellular-derived material and drastically helps to remove background in the reaction from undesired ligation products.
b. A variant of T4 RNA ligase 2 is used to increase the sensitivity as well as reduce the time needed to perform the ligation reaction.

### Step 2: PCR Amplification

Following the ATP-independent ligation of the RNA anchor to the 3′ end of cellular mRNAs in step 1, the anchor ligated polynucleotide can be used for amplification of the 3′ UTR/poly(A) region of the targeted mRNA using conventional RT-PCR with the following key improvements:
a. The reverse transcriptase (RT) and downstream amplification primer will not only target the anchor primer but will contain several bases of dT at its 3′ end (or dA for applications to detect poly(U) tails or another set of bases which represent the 3′ end of any polynucleotide sequence). This helps to ensure that the downstream amplification primer anchors precisely at the 3′ end of the adaptor for high accuracy in tail sizing.
b. Additionally, by adding a fixed pre-determined amount of downstream primer for both the RT and PCR amplification steps, we have dramatically reduced smearing of amplified bands in the assay (which is a problem with other PCR-based assays for poly(A) tail length).
c. We have already performed the requisite bioinformatics (and quality control testing) to determine optimal mRNA-specific upstream PCR primers that will be fully compatible with the Tₘ of the anchor primer and allow for accurate and efficient PCR amplification. This information for all current validated human mRNAs will be provided to researchers as a searchable table via the internet. This will remove the burden from the researcher to determine the best upstream primer to use for his/her desired mRNA, ensuring ease of use of the assay with minimal variability.

### Step 3: Sequencing or Sizing

PCR products can be cloned into suitable plasmid vectors using standard techniques. The AIR Adenylated Linker C sequence 5′-TTTAACCGCGAATTCCAG-3′ contains an EcoRI site (GAATTC) to enable cloning and subsequent sequencing. Thus, PCR products amplified with a gene specific primer that also has a restriction site can be cloned into an appropriately digested plasmid vector using standard cloning techniques. Alternatively, a quick PCR cloning kit can be used to clone the RACE products without using restriction digest. Also, the PCR fragments may be used in Next generation sequencing instruments for direct sequencing. Secondly, PCR products may be used to size the PCR fragments using standard techniques such as polyacrylamide or agarose gel electrophoresis, capillary electrophoresis, Mass Spectrometry, or any other technique to size the PCR fragements

### Sample Applications

1) This technology may be developed as a kit for poly(A) tail length and rapid amplification of cDNA ends (RACE) analysis. This analysis can be performed on any RNA with a 3′OH end.
2) This technology may be used to determine the difference of poly adenylated tail length between disease and non-disease state conditions for diagnosing disease.
3) This technology may be used to screen molecules that inhibit/deadenylate viral RNAs.
4) This technology may be used to examine miRNA regulation of poly(A) tail length and will be a vital kit in this analysis.
5) Adenylated linkers may be conjugated directly to antibodies, or indirectly to aptamers, in order to link two or more antibodies together for the purposes of a bi-epitopic recognition creating a significant improvement to proximity ligation assays.
6) The use of the adenylated linkers with a ligase enzyme may be used as a 3′-end RACE kit.
7) Attaching adenylated linkers to DNA ends.
8) Using the adenylated RNAs for detection by PCR.
9) Using adenylated small RNA for substrates in Reverse transcription and PCR detection assays.
10) Appending adenylated linkers with different sequences to polynucleotides derived from different cell preparations. Mixing the preparations together and amplifying the same genes out of the different cell preparations using different primer sets.
11) The use of the ligation approach to detect cleaved RNAs.
12) The use of real time PCR detection methods for quantifying poly(A) tail length using a Taqman assay containing a oligo dT probe. The larger the tail, the more signal may be detected because of the ability of more than one Taqman probe to anneal and generate a signal.
13) The use of controls that have defined length poly(A) tails for testing of the kits activity.

### BRIEF DESCRIPTION OF THE DRAWINGS

Advantages of the present invention will become apparent to those skilled in the art with the benefit of the following detailed description of embodiments and upon reference to the accompanying drawings in which:

FIG. 1 depicts a flow chart of the ALL-TAIL process overview;

FIG. 2 depicts experimental results of RNA ligation assays;

FIG. 3 depicts experimental results of a bridge ligation assay;

FIG. 4 depicts the results of an ALL-TAIL;

FIG. 5 depicts a comparison of the RNA sequence of the RNA compared directly with the sequence of the PCR product;

FIG. 6 depicts experimental results of studies of the poly(A) tail length of the circadian clock gene Arnt1 during light and dark cycles;

FIG. 7 depicts Poly(A) tail length changes in normal and cancer cells;

FIG. 8A-C depicts data collected from poly(A) tail length experiments; and

FIG. 8D depicts a schematic diagram of a non-gel based, mid-throughput assay to compare poly(A) tail length between samples in a 96-well format.

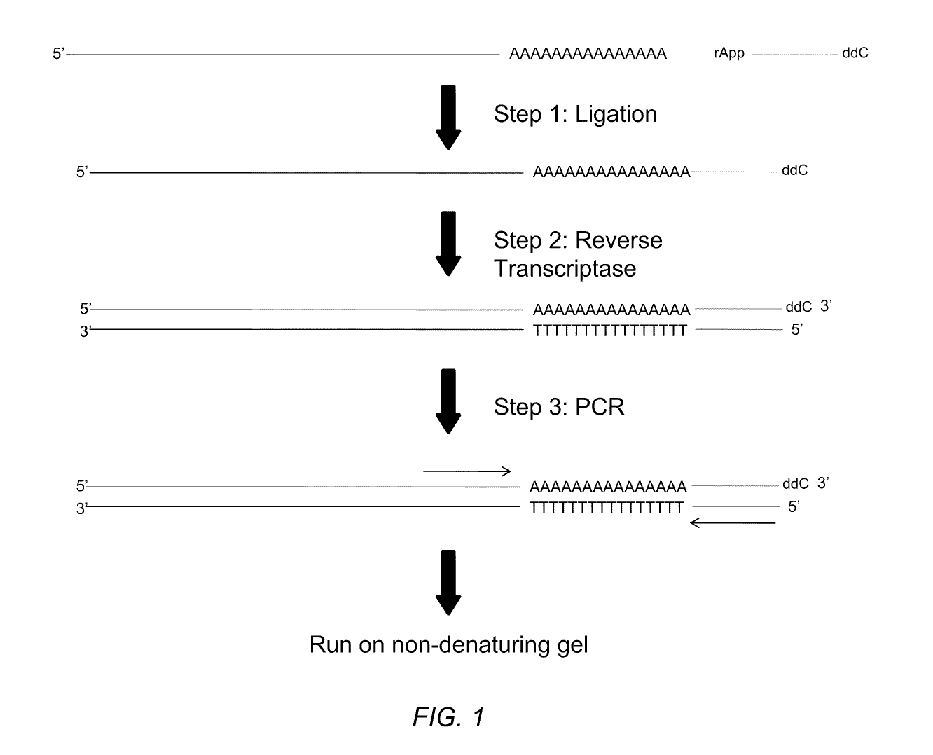

While the invention may be susceptible to various modifications and alternative forms, specific embodiments thereof are shown by way of example in the drawings and will herein be described in detail. The drawings may not be to scale. It should be understood, however, that the drawings and detailed description thereto are not intended to limit the invention to the particular form disclosed, but to the contrary, the intention is to cover all modifications, equivalents, and alternatives falling within the spirit and scope of the present invention as defined by the appended claims.

### DETAILED DESCRIPTION OF THE PREFERRED EMBODIMENTS

It is to be understood the present invention is not limited to particular devices or methods, which may, of course, vary. It is also to be understood that the terminology used herein is for the purpose of describing particular embodiments only, and is not intended to be limiting. As used in this specification and the appended claims, the singular forms “a”, “an”, and “the” include singular and plural referents unless the content clearly dictates otherwise. Furthermore, the word “may” is used throughout this application in a permissive sense (i.e., having the potential to, being able to), not in a mandatory sense (i.e., must). The term “include,” and derivations thereof, mean “including, but not limited to.” The term “coupled” means directly or indirectly connected.

### A. Isolation of Cellular RNA and Optimization of its Use in the ALL-TAIL

Total cellular RNA was prepared using standard Trizol (or equivalent) methodologies. Any RNA isolation procedure will yield transcripts that are fully compatible with the assay that we have developed. DNAse treatment of the samples is optional. Poly(A) tail assessment assays using total RNA samples that have been treated with RNAse-free DNAse (e.g., 1-5 units at 37° C. for 30 min. followed by phenol chloroform extraction and ethanol precipitation) may also undergo poly(A) sizing with minimal spurious amplification products.

The ALL-Tail assay is highly sensitive and requires only minimal amounts of input RNA to detect the poly(A) tail on an mRNA of interest. The exact amount of starting RNA needed is somewhat dependent on the relative abundance of the starting transcript. Serial dilutions of gel purified GemA60 RNA sample, starting from 10 μg through 0.5 μg have been assessed for the ability of the assay to detect poly(A) tail length of a synthetic RNA containing a 60 base poly (A) tail. Secondly we modulated the concentration of linker of A549 total RNA from 1 μg to 10 ng and analyzed the GAPDH poly(A) tail. Lastly, we modulated the level of linker with respect to input SinV 3′UTR synthetic RNA concentration. These experiments provided an important parameter for the assay kit and allows researchers to know a ballpark figure for the amount of starting material they will need in order to successfully assay the poly(A) tail length of their gene of interest.

### B. Details of Step 1: ATP-Independent Ligation of a 3′ Anchor Primer

Adenylated RNA oligomers that contain a pyrophosphate linkage at the 5′ end are efficient substrates for T4 RNA ligase. In addition, the use of pre-adenylated adapter RNA oligomers has been a significant improvement for the cloning and identification of small RNAs by minimizing many of the undesired products that can be obtained in an RNA ligation reaction that contains ATP. In an embodiment, this technology is utilized as the first step in a poly(A) tail assay. The use of a 3′ anchor primer (5′rAppTTTAACCGCGAATTCCAG/3ddC/3′ or any alternative to this sequence) offers the following advantages:
The 5′ rApp moiety will serve as the energy source for the reaction. Since ATP will be excluded from the reaction, this will ensure that the only molecule capable of ligating to a 3′ OH in the mixture will be this anchor primer.
The internal 17 mer sequence melting temperature ranges from 40° C. to 60° C. (see list below) but may extend beyond this range. The chosen sequence is optimized through bioinformatic predications and experimental verifications as outlined below. Modifying the internal sequence to increasing the Tm to ˜60° C. or greater, for example, allows the extension step in the PCR amplification part of the protocol to be eliminated, as well as to reduce non-specific PCR amplification products. Thus use of bioinformatic and experimental development to determine the optimal linkers provides a rational design of specific upstream target-mRNA specific primers that are of similar Tm to create an efficient and specific PCR amplification in the next step. In one embodiment, a set of adenylated linkers, each with a different melting temperature, may be presented in a kit for performing the ALL-Tail assay.
The placement of a dideoxy nucleotide at the 3′ position inhibits the oligo from ligating with itself or any other cellular oligo, therefore inhibiting the generation of multimers that would interfere with the precise determination of poly(A) tail length.

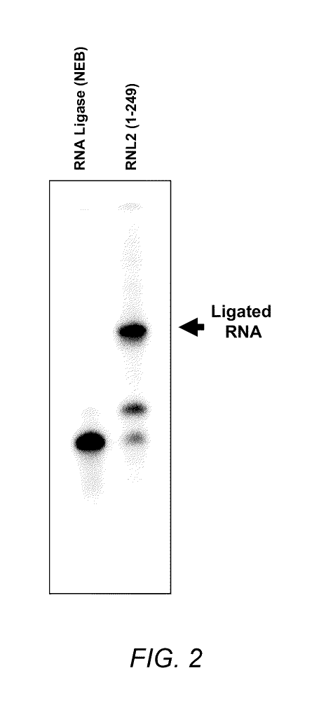

In an embodiment, a variant of T4 RNL2 that ligates RNA >100 fold more efficiently than the conventional preparations of RNA ligase that we have tested to date is used in the assay. A variant of T4 RNL2 that may be used, AIR® Ligase, is commercially available from Bioo Scientific Corporation, Austin Tex. FIG. 2 depicts a kinased RNA oligomer that was incubated with either commercial RNA ligase (NEB) [20U] or the variant of a T4 RNA ligase isoform N terminal fragment (RNL2 1-249). Samples were incubated at 16° C. for 3 hrs and analyzed for ligation efficiency on a 5% denaturing acrylamide gel.

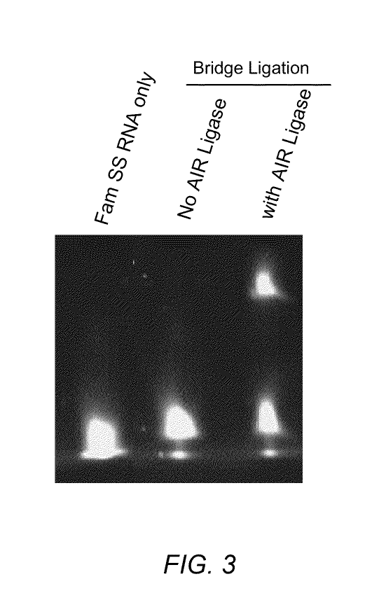

In some embodiments, a bridging oligo can assist the RNA ligation reaction and may be a component of the assay when using larger amounts of input RNA. This bridging oligo may be an exact complement of the linker and also have a run of T residues to ensure hybridization to the polyA tail of mRNA. The run of T residues will also subtract out non-polyA RNA from being used as ligation substrates. FIG. 3 depicts a bridge ligation assay in which 0.5 μg of an equimolar mix of a 17mer 5′-FAM labeled RNA (bold), AIR™ adenylated oligonucleotide (A) and a complementary bridge oligo (B) along with AIR Ligase and buffer in a total reaction volume of 20 μL. The mixture was incubated at 37 C for 30 minutes and the material was run, along with a no enzyme control, on a 20% denaturing polyacrylamide gel.

(A)
FAM-5′-CUUUUUCCCCCUCGCGGCTGTAGGCACCATCAAT/3ddC/3′
(B)
3′-GAAAAAGGGGGAGCGCCGACATCCGTGGTAGTTA -5′

Example sequences are listed below that may be used to generate adenylated linkers containing different Tm's for the polyA tail length and position analysis assay.

| | | Energy | Energy | |
|---|---|---|---|---|
| Primer | Tm | (duplex) | (fold) | GC % |
| CGAATTACGACTTGCGTATAGA | 50.56 | −8.00 | −1.90 | 40.91 |
| CGACCGATAATTATCTATCCGC | 50.79 | −11.80 | −1.00 | 45.45 |
| CGTTAAACGATTGATTACGTCG | 50.79 | −8.60 | −0.90 | 40.91 |
| CCGACGTATAACGCATACTATC | 50.81 | −4.90 | −0.80 | 45.45 |
| CGTAATCGCTAACTTACGAGTC | 51.24 | −7.70 | −2.60 | 45.45 |
| CGGATTACGTATATCGTTACGC | 51.46 | −9.90 | −2.50 | 45.45 |
| TACGACGCTAACTTATCCGATC | 51.89 | −2.40 | 0.00 | 45.45 |
| CGAATTACGACCGGAGCTATAA | 52.10 | −8.10 | 0.00 | 45.45 |
| ACGGTCGATAACTCATATCGTG | 52.13 | −8.00 | −2.60 | 45.45 |
| CGAAACGTTATCGCGTACTAAG | 52.14 | −9.10 | −2.10 | 45.45 |
| TATCGCTACCGTAGTTAATCGC | 52.22 | −11.50 | 0.00 | 45.45 |
| TAACGTACGAACCGATGTAGTG | 52.30 | −9.60 | −0.80 | 45.45 |
| CGAATTTAGCGCGACTCTTATG | 52.37 | −8.90 | 0.00 | 45.45 |
| ACTAATCGCGAATCGGGTTATC | 52.68 | −13.40 | −1.50 | 45.45 |
| TCGCTAAACCGTTAGATCGAAC | 52.87 | −7.60 | −1.30 | 45.45 |
| TAACGTACGAACCGAACTTACG | 52.88 | −9.60 | −0.50 | 45.45 |
| CGACTAAATCGCGACCCATAAT | 52.94 | −9.50 | 0.00 | 45.45 |
| TAGTCGAACGATCGTTACAACG | 52.97 | −16.00 | −1.10 | 45.45 |
| CGCTACGTAACGCCAATTTAAC | 53.07 | −8.70 | −0.30 | 45.45 |
| TCGACTAAACGTGCCGAATTAG | 53.13 | −6.30 | −0.80 | 45.45 |
| GCGATTAACGAGACGACGTATT | 53.33 | −5.50 | −0.80 | 45.45 |
| ACTTATCGACGAGTCAGCGTAT | 53.34 | −11.50 | −1.10 | 45.45 |
| CGTACGAATCTACAAGAGACGC | 53.36 | −8.60 | −0.90 | 50.00 |
| TCGACTAAACGACCCAATCGTA | 53.44 | −7.00 | −2.00 | 45.45 |
| TATCGCTACCGCGTAGATTGAT | 53.44 | −11.70 | −2.30 | 45.45 |
| CGATCAACGTATATGACGACGG | 53.49 | −13.00 | −1.40 | 50.00 |
| CGATCGACTTACGCCGTAATTT | 53.55 | −9.50 | 0.00 | 45.45 |
| TATGCGTAACGTACCGCTCTTA | 53.61 | −13.70 | −2.40 | 45.45 |
| ATCGACGCTTATCATACCGGTT | 53.86 | −8.90 | 0.00 | 45.45 |
| TTAGCGTATCGACCCAATCGTT | 54.08 | −5.90 | 0.00 | 45.45 |
| ATCGTACGATACGCCTATTCGG | 54.17 | −13.80 | −1.30 | 50.00 |
| CTATCCGAACGATAAGGCCGAA | 54.23 | −9.70 | −1.20 | 50.00 |
| CGCGACTAAATGTTCGAGATCG | 54.27 | −6.30 | −1.10 | 50.00 |
| CGAATTACGACGGTCATTGAGC | 54.40 | −10.50 | 0.00 | 50.00 |
| ATCGACGCTAGCGGTGAATTTA | 54.43 | −19.60 | −2.50 | 45.45 |
| TACCGTAACGCGATCCTTTACC | 54.45 | −9.60 | −0.40 | 50.00 |
| TCGATTTAGCGACGTCTTACCC | 54.47 | −10.50 | −0.70 | 50.00 |
| TAACGTACGACGCAAGGGATAC | 54.48 | −9.60 | −1.80 | 50.00 |
| TAATCGTACGGGTTATGGCGTC | 54.51 | −10.60 | −1.20 | 50.00 |
| ATCGTACGATACTCGATCCGGA | 54.53 | −13.80 | −1.10 | 50.00 |
| TACGATCGGAACGGAAATACCG | 54.54 | −10.20 | −1.70 | 50.00 |
| TACGACGCTAATACCTCGGCTA | 54.70 | −5.49 | −1.70 | 50.00 |
| TATCGCTACCGTACGCTCAGTA | 54.73 | −10.60 | −0.70 | 50.00 |
| ATCGAACCGTAATAACGCGACA | 54.74 | −7.30 | −1.40 | 45.45 |
| ATCGATCGTTATACGTAGCGCC | 54.78 | −10.50 | −2.30 | 50.00 |
| GCGAACCGATAGTACGTCCAAT | 54.78 | −6.60 | −1.00 | 50.00 |
| TATGCGTAACGTAGGCGAAGAC | 54.83 | −11.60 | −1.50 | 50.00 |
| ACGGTCGATAACGCGAAATTCT | 54.98 | −8.00 | −1.20 | 45.45 |
| ATCGATCGTTAAACGACGCACT | 55.01 | −12.60 | −2.40 | 45.45 |
| CGATCGACTTACGGCGAAGATT | 55.16 | −9.20 | −1.60 | 50.00 |
| CGAATTACGACGTGATAGCCGT | 55.17 | −12.10 | −0.70 | 50.00 |
| TAATCGTACGGCGCCAATTTGA | 55.19 | −15.70 | 0.00 | 45.45 |
| AGCGTAAACCGTTCGAGAAGTC | 55.20 | −7.10 | −0.50 | 50.00 |
| CTACGGAACGACGATCGAGTTC | 55.22 | −15.20 | −1.30 | 54.55 |
| CGTTAAACGATGGCGAACAACC | 55.33 | −7.40 | −2.20 | 50.00 |
| ATACGACGTTAACGTAACGGCC | 55.35 | −14.40 | −2.40 | 50.00 |
| CTAATCGAACGGGACGAGAACG | 55.44 | −9.10 | −0.60 | 54.55 |
| ACTAATCGCGATGGTCCGTAGT | 55.46 | −13.30 | −1.50 | 50.00 |
| TACGTAACGGCGCACTATAGGT | 55.49 | −8.90 | −0.80 | 50.00 |
| TAATCGTACGGGACGTGATCGT | 55.51 | −10.60 | −0.80 | 50.00 |
| TACGATCGGAACGGGTAACCTC | 55.74 | −10.20 | −1.60 | 54.55 |
| TAATCGTACGGTTCGCGAGAGT | 55.79 | −13.60 | −2.40 | 50.00 |
| CGTACAATCGACGCGATCTACG | 55.96 | −13.70 | −2.40 | 54.55 |
| TCGACGTAACGACGCTAGAACA | 56.02 | −7.60 | −1.10 | 50.00 |
| TAACGTACGACAGGCGATCGAA | 56.06 | −9.60 | −1.20 | 50.00 |
| GAACGTACGTAATATCGCCCGC | 56.14 | −11.80 | −1.80 | 54.55 |
| TACCGTAATCGCGCAATAGGCT | 56.38 | −9.70 | −0.70 | 50.00 |
| CGACGTTATACTCGCCGATTGC | 56.40 | −6.40 | −1.10 | 54.55 |
| CGCGACTAAATCGGGTACCAGT | 56.59 | −11.60 | −1.60 | 54.55 |
| CGAATTACGACGCGCGATAAGG | 56.74 | −13.10 | 0.00 | 54.55 |
| ACTAATCGCGATCCTCGTTCGG | 56.75 | −10.90 | −1.50 | 54.55 |
| GCGCTACGTAACGTACATTCGC | 56.93 | −14.00 | −2.00 | 54.55 |
| CTATCCGAACGCGACCTGTCAG | 57.27 | −7.70 | −0.30 | 59.09 |
| TATGCGTAACGCCCTAACAGCG | 57.50 | −11.80 | −1.40 | 54.55 |
| CGCTAATCGACATCCGCACAGT | 57.51 | −5.90 | −0.10 | 54.55 |
| CTACCGTAACGCGACACATCCC | 57.52 | −8.30 | −0.40 | 59.09 |
| TATACGTCCGAGGCGCTACGTT | 57.73 | −13.70 | −1.90 | 54.55 |
| TAACGCTACGCATGGTTCGTCG | 57.78 | −9.40 | −0.60 | 54.55 |
| TCGACTAAGCGCGCCGATTTAC | 57.84 | −12.90 | 0.00 | 54.55 |
| CGATCGACTTACCGCCCGTAGA | 58.06 | −8.80 | −0.50 | 59.09 |
| TCTAACCGACGAACGATCCCGG | 58.23 | −8.90 | −1.10 | 59.09 |
| TCGATTTAGCGCCGTCGTCTGA | 58.50 | −12.10 | −1.40 | 54.55 |
| CGTAACCGACTACCTTTGCGCG | 58.60 | −8.50 | −0.90 | 59.09 |
| CGCTAATCGACCCGCCGATACT | 58.68 | −6.70 | −2.50 | 59.09 |
| CCGACTAACGGGCTAAGTGCGA | 58.74 | −13.40 | −2.70 | 59.09 |
| TCGTACGTTAAGGCCCGGAACG | 58.99 | −14.10 | −0.70 | 59.09 |
| CTACGGAACGAACACGCCGAGT | 59.25 | −9.45 | −2.40 | 59.09 |
| GCGAACCGATAGAACCGCGAGT | 59.37 | −7.80 | −2.30 | 59.09 |
| CTACCGTAACGGACACCTCGCG | 59.38 | −13.20 | −1.60 | 63.64 |
| AGCGTAAACCGTCTGCGACTCG | 59.56 | −10.90 | −1.80 | 59.09 |
| GACGTAACCGAACCGATGCCCC | 59.87 | −3.90 | 0.00 | 63.64 |
| TACGCCTAACGCACGCTCAAGC | 60.12 | −5.60 | −0.70 | 59.09 |
| ACGTCTAAACGCCGTGCGCATT | 60.30 | −11.90 | −1.00 | 54.55 |
| CGACTAACGCTCCCGCTCGAGA | 60.42 | −9.90 | −1.30 | 63.64 |
| CGTAACGATTATGCGGGCCGCC | 60.85 | −17.80 | −1.60 | 63.64 |
| AACGTACCGGACGCATCGCAGT | 61.11 | −13.10 | −0.50 | 59.09 |
| TCGACGTAACGCGCAGGCCTAC | 61.47 | −11.10 | −0.90 | 63.64 |
| CGCCTAACGTACCCCGCGGAGT | 62.80 | −13.90 | −1.40 | 68.18 |
| CTACGGAACGAGGCCCCGCGCA | 65.45 | −13.70 | −1.70 | 72.73 |

In summary, the current assay overcomes the major drawbacks in previous assay methodologies for poly(A) tail length assessment.

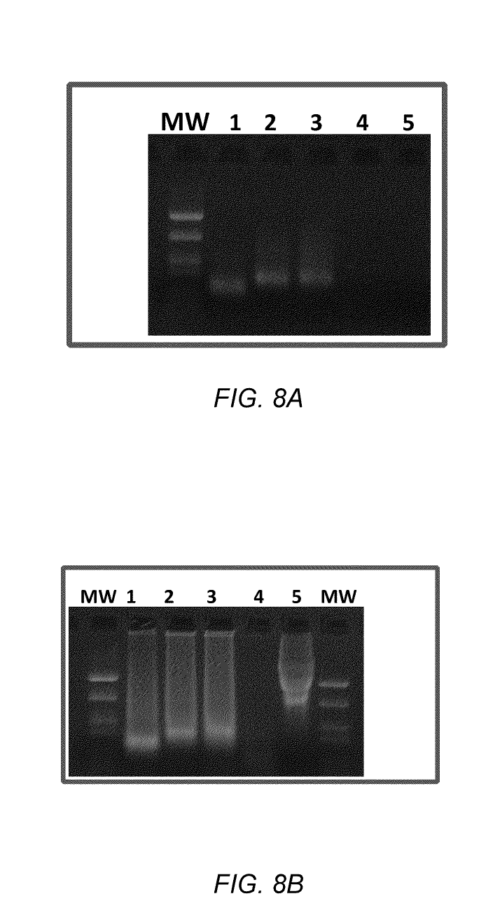

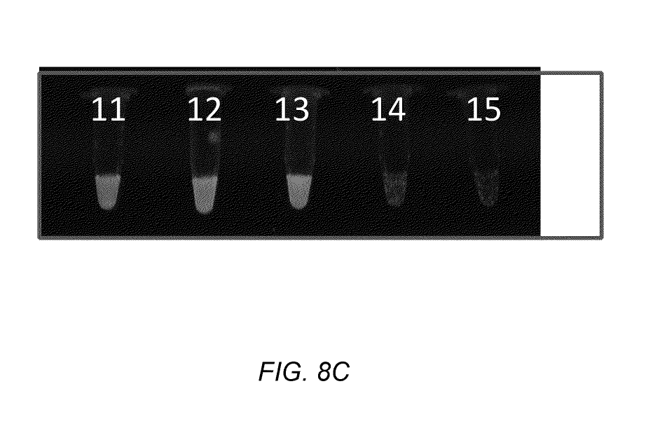

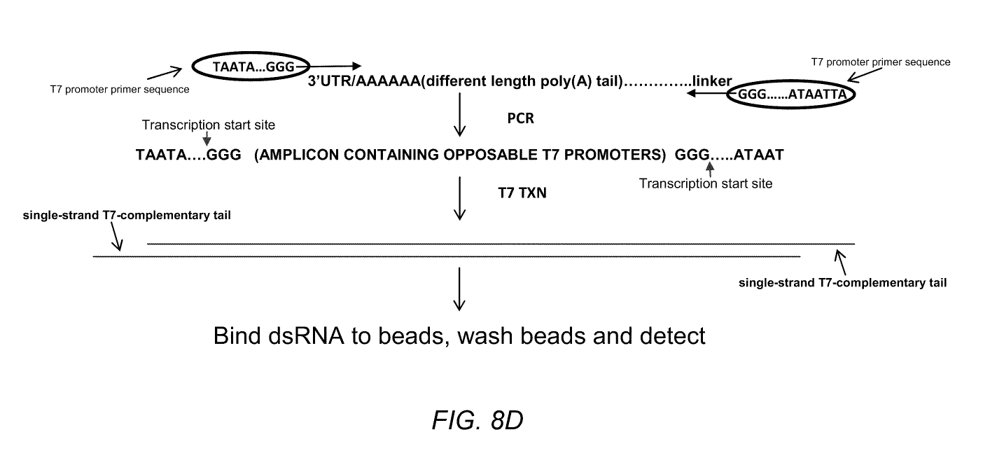

In an embodiment, outlined in the FIG. 8D, ligation of preadenylated linkers to the 3′ end of an RNA sample, followed by RT-PCR (reverse transcription-polymerase chain reaction) using gene-specific forward and linker-complementary reverse primers that contain the T7 promoter sequence at their 5′ ends, is performed so that after reverse transcription and PCR, the resulting amplicon contains opposable T7 promoters. The unpurified amplicon is then used as template in T7 in vitro transcription reactions to generate double-strand RNA (purification to remove unincorporated T7-PCR primers is not needed since they are single-stranded and so not recognized by T7 polymerase). Multiple copies of complementary transcripts are produced from each strand of each amplicon. The length of each transcript (which is dependent on length of poly(A) tract) determines the amount of double-strand-specific dye (e.g., SYBR Green) that binds to generate signal, so that incorporation of the dye label is proportional to length of poly(A) tract in the amplicon. Feasibility of the assay is shown by the data in FIGS. 8A-8C, using a tube format. Reactions may be carried out in 96-well plates and the level of dye incorporation in each sample may be determined in standard plate readers or in real-time PCR instruments.

Two potential related technical problems with this approach are distinguishing between a given signal being due to more templates with shorter poly(A) tracts vs fewer templates with longer tracts; and selecting for transcripts that have copied through the whole poly(A) tract (which is essential for the signal to reflect length of poly(A) tract, as opposed to detecting signal from truncated products). To deal with both of these issues, beads coupled to a capture oligo having the sequence of the T7 promoter may be used to recover constant amounts of double-strand RNA products that represent only full-length labeled transcripts. Hybridization of the dsRNAs to the beads may be carried out under bead-limiting/target excess conditions, so that capture probe on many of the beads is bound to a dsRNA target. This may help ensure equal numbers of captured dsRNA molecules per bead, so that signal recovered per bead will reflect the length of the poly(A) tract, and not be affected by the number of labeled transcripts produced in the transcription reaction. The experimental design also ensures that only full-length transcripts are captured. This is due to the fact that transcription originates at the G located at the 17^{th} base from the 5′ end of the 20-base T7 consensus promoter sequence; for a double-stranded DNA template with a T7 promoter at each end, the transcript from each strand initiates at the 17^{th} base and “runs off' after transcribing through the entire T7 promoter at the other end. After the complementary transcripts from each strand have hybridized, the resulting product is a double-stranded RNA molecule with 16 base single-stranded “tails”, whose sequences are the complement of the T7 promoter. The single-strand tail may be extended by adding extra bases (for example a 5 base bar-code) upstream of the T7 promoter. The capture oligo attached to the beads is the T7 promoter sequence. Stalling of the T7 polymerase while copying through long homopolymeric regions (A's in one direction, from one strand, and U's from the other strand) will generate transcripts that will not have the promoter-complementary sequence that is read only from full-length transcripts, and so these will not be captured onto the bead. This consideration means that truncated transcripts will not generate signal in the assay. This assay may be used as a screening tool; samples with fluorescent signals that stand out as being different from the majority of samples can be further characterized by running the amplicons on a gel to confirm the size and number of bands.

In another embodiment, calibration standards are used against which unknown samples are compared to allow quantitative assessment of 3′UTR/poly(A) amplicon lengths in the non-gel based assay. The calibration standards may be generated using recombinant plasmids with poly(A) tracts of different lengths, for example 15, 50, 100, and 150 bases. An assay kit may, in some embodiments, include one or more of: pre-adenylated linkers, calibration standards, MMLV reverse transcriptase, RT buffer, dNTPs, beads with capture oligos, Universal reverse primer with T7 promoter, T7 RNA polymerase and buffer, and rNTPs. Users may need to supply SYBR Green or other appropriate double-strand-specific nucleic acid stain, gene-specific forward primers with T7 promoters, and equipment for separation of the beads and detection of the SYBR-stained dsRNA. Magnetic separation stands or microtiter plate-compatible microfuges are available for use in capturing bead-associated products from single reactions and also in 96-well plate formats. The detection step may be carried out on fluorescent plate readers or qPCR thermalcyclers, common equipment to which most labs have access. Judicious design of the forward primers may be used for assessing associations between different 3′UTR/poly(A) sequences and alternative mRNA coding region isoforms. An isoform-specific forward primer (without the T7 promoter) would be used in an initial amplification step, along with a reverse primer targeted to the linker sequence ligated to the end of the mRNA. This PCR step will select only the isoform of interest. Using that amplicon as template, a second round of PCR can be carried out using a T7-containing forward primer that binds near the end of the coding sequence, along with a reverse primer that is identical to the original except for containing the T7-promoter sequence at the 5′ end, to create the template with opposable T7 promoters to use for the non-gel based assay.

### C. Details of Step 2: RT-PCR Amplification and Product Analysis

The second and final step of the protocol for the assay that we have developed involves fairly standard RT-PCR amplification of terminal portion of the 3′UTR and poly(A) tail of the target RNA that has been ligated to the anchor primer. However there are two key aspects of the protocol to our invention which make it somewhat unique from standard PCR amplification:
The downstream RT and PCR primer are complementary to the anchor sequence alone or the anchor sequence plus an additional dT residue at the 3′ end (or another sequence) to provide added specificity for polyadenylated RNAs which contain an anchor primer that has been directly ligated to the 3′ end of the tail. The assay is also adaptable to search for RNAs that have been polyuridylated by the recently recognized family of poly(U) polymerases inside the cell by attaching 3′ dA residues to the PCR primer rather than dT. While the RT reaction step will follow standard methods, we have determined an amount of primer to be added once that is sufficient for both the RT and PCR amplification steps. Addition of this pre-determined amount eliminates the smearing of product bands that is routinely observed when one attempts to amplify homopolymer stretches like a poly(A) tail.
The upstream primer is determined via our detailed bioinformatics analysis to identify mRNA-specific primers that meet the following criteria: (a) are within 200 bases of the start of the poly(A) tail; (b) have a Tm that is compatible with the Tm of the primer that hybridizes to the anchor RNA.
   This latter bulleted aspect may be supported by a complete table of human (and subsequently other species, organisms) mRNA-specific oligos that fit both criteria A and B. This table may be an invaluable resource for the ALL-tail kit invention as it would give all researchers full confidence in the PCR amplification step of the protocol. The extensive sequence data on multiple organisms at the NCBI may provide all of the requisite information for this aspect of the invention. One last note to illustrate the utility of this step is to specifically point out the two key benefits of using design primer sets with a Tm of 60° C. First the use of primer sets with a Tm of 60 C would minimize non-specific PCR products due to the high hybridization temperatures used in the assay. Second, using temperatures of 60° C. in the annealing step of the PCR reaction, allows the omission of the usual 72° C. extension step of the PCR reaction step, thereby increasing the speed of the assay.

In summary, in Step 2 the RT-PCR phase of the poly(A) tail assay is optimized with regard to the development of effective RT-PCR primers with optimal Tms (as high as is feasible) and minimized the time-to-data.

Recent evidence suggests that viruses have evolved a means of specifically stabilizing their RNAs in cells during infection. If one could interfere with the virus' ability to stabilize its RNAs, one can perhaps develop novel antiviral therapeutics. The All-Tail assay, since it assesses a fundamental aspect of mRNA decay (deadenylation is the first step in the turnover of many cellular mRNAs), may be used as a screening tool to identify small molecules from chemical libraries that block a virus' ability to maintain the stability of its mRNAs.

In one embodiment, a method of determining the effect of a molecule on the regulation of poly(A) tail length in viral RNA includes: forming a mixture of the molecule with viral RNA; separating the viral RNA from the mixture; ligating an oligonucleotide sequence to the 3′ end of the viral RNA molecule; reverse transcribing the ligated viral RNA molecule using a primer complementary to all or part of the ligated oligonucleotide sequence; amplifying the transcribed viral RNA to produce a mixture comprising multiple copies of the viral RNA molecule; and determining the poly(A) tail length of the viral RNA molecule. The determined poly(A) tail length of the viral RNA is compared to the poly(A) tail length of the viral RNA prior to mixing the viral RNA with the molecule. If the length of the poly(A) tail is shorter than the unreacted viral RNA poly(A) tail length, the molecule may be considered to be effective in inhibiting the virus from stabilizing the viral RNA.

Recent evidence has indicated that miRNAs can regulate gene expression by mediating changes in poly(A) tail length. To date, assessing changes in poly(A) tail length has been a cumbersome process for the reasons stated above. Thus the All-Tail assay provides a novel and highly effective approach to determine the broad influences of specific miRNAs on poly(A) length.

In one embodiment, a method of determining the effect of miRNA on the regulation of poly(A) tail length in RNA includes: forming a mixture of a miRNA molecule with RNA molecules; separating the RNA molecules from the mixture; ligating an oligonucleotide sequence to the 3′ end of one or more RNA molecules; reverse transcribing the ligated RNA molecules using a primer complementary to all or part of the ligated oligonucleotide sequence; amplifying the transcribed RNA molecules to produce a mixture comprising multiple copies of the RNA molecules; and determining the poly(A) tail length of the RNA molecules. The determined poly(A) tail length of the RNA is compared to the poly(A) tail length of the RNA prior to mixing the RNA with the miRNA. If the length of the poly(A) tail is substantially different than the unreacted RNA poly(A) tail length, the miRNA may be considered to be effective in regulating the RNA poly(A) tail length.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

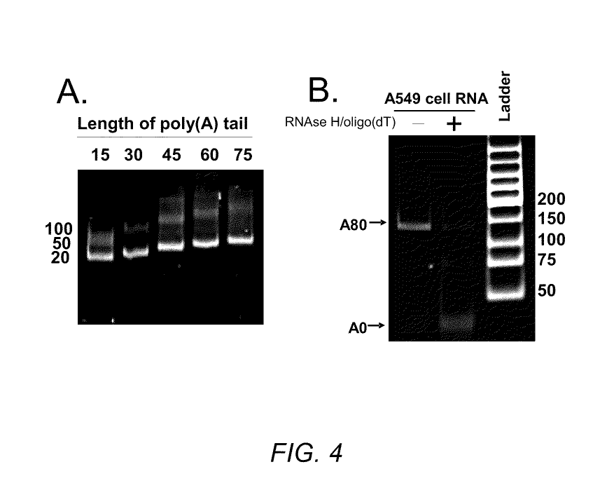

We used the ALL-Tail method to analyze its effectiveness on synthetic RNA containing defined length poly(A) tails of 15, 30, 45, 60 and 75 bases in length (panel A) or GAPDH (panel B), as shown in FIG. 4. RNA ligase storage buffer: 10 mM Tris-HCl (pH 7.5), 100 mM NaCl, 0.1 mM EDTA, 0.1 mM DTT and 50% glycerol. 1× RNA ligase Reaction buffer: 50 mM Tris-HCl (ph 7.5), 2 mM MgCl₂, 1 mM DTT. In panel A, plasmids were generated that contain poly(A) tail lengths ranging in size from 15 to 75 bases in length by cloning hybridized oligonucleotides into the multiple cloning site of pGem 4. The plasmid was linearized using Nsi1 and transcribed using the SP6 transcription kit (Ambion). RNA with a defined length tail was gel purified and used in the ALL-Tail assay. RNA was ligated by mixing 10 ng of in vitro transcribed RNA containing defined length tails with 4.5 μL of a 10 μM solution of adenylated linker (5′rAppTTTAACCGCGAATTCCAG/3ddC/3′), 2.4 μL of 50% PEG, 1 μL of RNA ligase buffer and 1 μL of AIR Ligase. The reaction was incubated at room temperature for 3 hours. The ligated RNA was next used for reverse transcription by adding the following together, 1 μL of MMuLVRT, 2 μL of 10× RT Buffer, 1 μL of Ligated RNA, 1 μL of RT/PCR primer which was 50 μM (5′-CTGGAATTCGCGGTTAAA-3), 1 μL of dNTP mix (20 mM), 1 μL RNAse Inhibitor and 12 μL RNase free ddH₂O. The sample was incubated at 25 C for 10 minutes, then incubated at 42 C for 60 minutes. Lastly, it was denatured at 95 C for 10 minutes. The RT sample (2.5 μL) was next used for PCR amplification by mixing, 10 μL of PCR MasterMix, 1 μL of RT/PCR primer and ddH₂O to bring the total volume to 50 μL. The samples were amplified by using a program that has 1 cycle of 94 C for 3 min. and 30 cycles of (94° C. for 30 seconds, 50° C. for 45 seconds and 72° C. for 60 seconds). One final cycle of 72° C. for 5 minutes was performed before storing at 4° C. or running on a gel. The products from the PCR reaction were analyzed on a 10% acrylamide gel which was stained with ethidium bromide. Defined length bands representing the defined length poly(A) tail were effectively detected (Panel A). The GAPDH mRNA poly(A) tail was next assessed with the ALL-Tail assay using the reaction conditions listed above using 1 μg A549 total RNA rather than the control template RNA with defined length tails. The lane marked ‘−’ was not pre-treated with RNAse H/oligo(dT) prior to analysis; the lane marked “+” was pre-treated. Following the ALL-TAIL procedure, the PCR products were analyzed on a 10% acrylamide gel. The data shown in panel B demonstrates the ALL-TAIL assay accurately detects the poly(A) tail of the GAPDH mRNA. Based upon the forward and reverse primers locations within the amplicon the poly(A) tail of GAPDH is 80-90 bases in length which is consistent with that published.

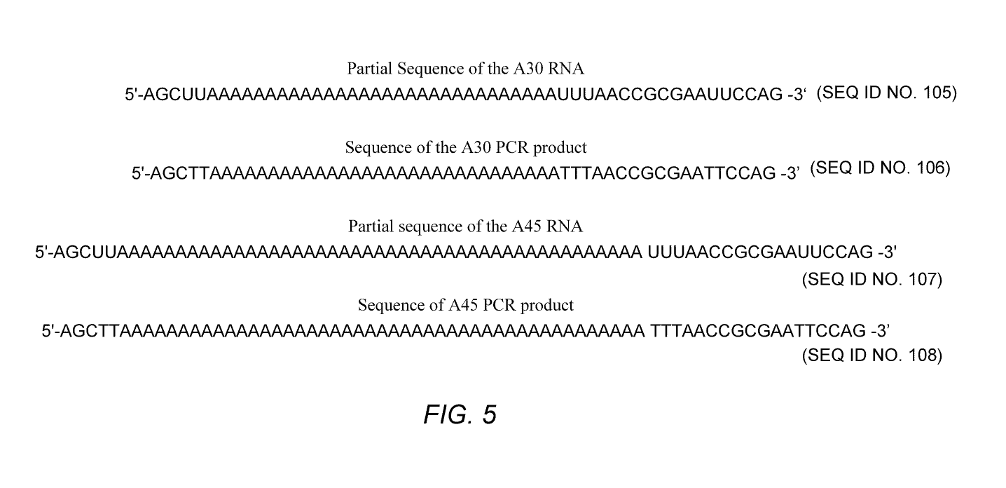

A validation of the amplicons from the control transcripts is depicted in FIG. 5. RNA derived from the A30 and A45 control transcripts was ligated by mixing 10 ng of in vitro transcribed RNA containing defined length tails with 4.5 μL of a 10 μM solution of adenylated linker (5′rAppTTTAACCGCGAATTCCAG/3ddC/3′), 2.4 μL of 50% PEG, 1 μL of RNA ligase buffer and 1 μL of AIR Ligase. The reaction was incubated at room temperature for 3 hours. The ligated RNA was next used for reverse transcription by adding the following together, 1 μL of MMuLVRT, 2 μL of 10× RT Buffer, 1 μL of ligated RNA, 1 μL of RT/PCR primer which was 50 μM (5′-CTGGAATTCGCGGTTAAA-3), 1 μL of dNTP mix (20 mM), 1 μL RNAse Inhibitor and 12 μL RNase free ddH₂O. The sample was incubated at 25° C. for 10 minutes, then incubated at 42° C. for 60 minutes. Lastly, it was denatured at 95° C. for 10 minutes. The RT sample (2.5 μL) was next used for PCR amplification by mixing, 10 μL of PCR MasterMix, 1 μL of RT/PCR primer and ddH₂O to bring the total volume to 50 μL. The samples were amplified by using a program that has 1 cycle of 94° C. for 3 min. and 30 cycles of (94° C. for 30 seconds, 50° C. for 45 seconds and 72° C. for 60 seconds). One final cycle of 72 C for 5 minutes was performed before storing at 4° C. or running on a gel. The products from the PCR reaction were analyzed on a 10% acrylamide gel which was stained with ethidium bromide. The comparison of the RNA sequence that is predicted from the DNA clone containing a 30 and 45 base poly(A) tail is compared directly with the sequence of the PCR product. The sequencing data comparison demonstrates that the ALL-Tail assay is generating PCR products representing the distinct tail length of the input RNA thus validating the procedure and the sequence integrity of the bands in the gel. This also sets the stage for further validation of the poly(A) tail length and poly(A) tail site selection by the relatively inexpensive Sanger sequencing.

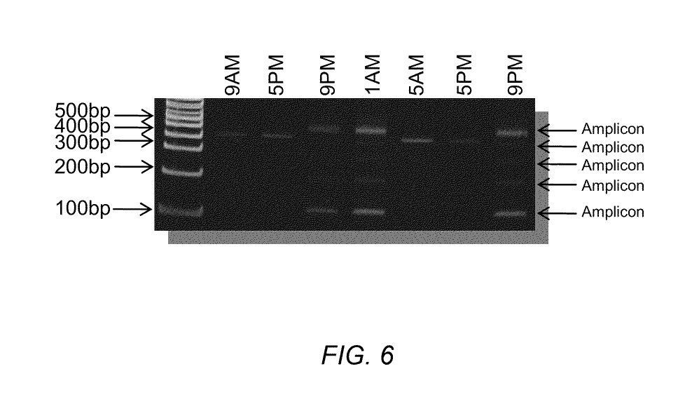

In FIG. 6, we used the ALL-Tail kit to assess poly(A) tails for a gene related to circadian rhythm. In this experiment, RNA was extracted from mouse white blood cells obtained at 4 hour intervals over a 24 hour time-course. RNA was ligated by mixing 10 ng of in vitro transcribed RNA containing defined length tails with 4.5 μL of a 10 μM solution of adenylated linker (5′rAppTTTAACCGCGAATTCCAG/3ddC/3′), 2.4 μL of 50% PEG, 1 μL of RNA ligase buffer and 1 μL of AIR Ligase. The reaction was incubated at room temperature for 3 hours. The ligated RNA was next used for reverse transcription by adding the following together, 1 μL of MMuLVRT, 2 μL of 10× RT Buffer, 1 μL of Ligated RNA, 1 μL of RT/PCR primer which was 50 μM (5′-CTGGAATTCGCGGTTAAA-3), 1 μL of dNTP mix (20 mM), 1 μL RNAse Inhibitor and 12 μL RNase free ddH₂O. The sample was incubated at 25 C for 10 minutes, then incubated at 42 C for 60 minutes. Lastly, it was denatured at 95 C for 10 minutes. The RT sample (2.5 μL) was next used for PCR amplification by mixing, 10 μL of PCR MasterMix, 1 μL of RT/PCR primer and ddH₂O to bring the total volume to 50 μL. The samples were amplified by using a program that has 1 cycle of 94 C for 3 min. and 30 cycles of: 94 C for 30 seconds; 50 C for 45 seconds; and 72 C for 60 seconds. One final cycle of 72 C for 5 minutes was performed before storing at 4 C or running on a gel. The products from the PCR reaction were analyzed on a 10% acrylamide gel which was stained with ethidium bromide. The Arnt1 gene showed variation in poly(A) tails, with the presence of several discrete amplicons that varied between samples. We will sequence the set of 3′UTR/poly(A) amplicons from the circadian study, they will be excised from a high-resolution acrylamide gel, cloned or sequenced directly using the forward and reverse primer sets. The sequencing data from the ARNT1-derived amplicons will not only serve to further validate our method, but will also generate biologically interesting data that will be useful for marketing the ALL-Tail kit.

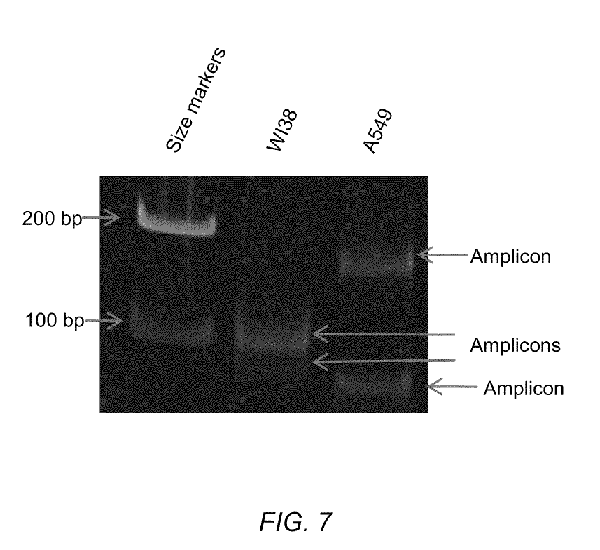

In FIG. 7, RNA from a normal cell line (WI38) and a cancer cell line (A549) were extracted using BiooPure RNA isolation reagent. RNA samples were used for ALL-Tail as follows: RNA was ligated by mixing 1 μg total RNA with 4.54 of a 10 μM solution of adenylated linker (5′rAppTTTAACCGCGAATTCCAG/3ddC/3′), 2.4 μL of 50% PEG, 1 μL of RNA ligase buffer and 1 μL of AIR Ligase. The reaction was incubated at room temperature for 3 hours. The ligated RNA was next used for reverse transcription by adding the following together, 1 μL of MMuLVRT, 24 of 10× RT Buffer, 1 μL of Ligated RNA, 1 μL of RT/PCR primer which was 50 μM (5′-CTGGAATTCGCGGTTAAA-3), 1 μL of dNTP mix (20 mM), 1 μL RNAse Inhibitor and 124 RNase free ddH₂O. The sample was incubated at 25 C for 10 minutes, then incubated at 42 C for 60 minutes. Lastly, it was denatured at 95 C for 10 minutes. The RT sample (2.5 μL) was next used for PCR amplification by mixing: 10 μL of PCR MasterMix, 1 μL of RT/PCR primer and ddH₂O to bring the total volume to 50 μL. The samples were amplified by using a program that has 1 cycle of 94 C for 3 min. and 30 cycles of: 94 C for 30 seconds; 50 C for 45 seconds; and 72 C for 60 seconds. One final cycle of 72 C for 5 minutes was performed before storing at 4 C or running on a gel. The products from the PCR reaction were analyzed on a 10% acrylamide gel which was stained with ethidium bromide.

A non-gel based, mid-throughput assay to compare poly(A) tail length between samples using a 96-well format was developed. The assay is based on detection of double stranded RNA produced by T7 transcription of PCR amplicons with opposable T7 promoters. Feasibility of the method is shown by the experiments depicted in FIGS. 8A-8C, which was carried out in microfuge tubes and assessed on a UV transilluminator. This assay may also be carried out in a 96-well microtiter plate with detection on a plate-reader. The bead-capture step may be carried out under controlled conditions by adding a constant number of beads to each sample, so that the same number of bead-coupled T7 capture oligos is added to each sample, and the number of beads/capture oligos added is controlled such that the capture oligos are in all cases less than the number of double-strand RNA target molecules in each sample. That is, the capture oligos are the limiting species in the hybridization reaction. This ensures that all the capture oligos will be hybridized to dsRNA targets, so that the same number of dsRNA targets will be captured for each sample. This consideration is important to ensure that the signal generated in each reaction is proportional to the length of dsRNA molecules for each sample, and not to the number of dsRNA target molecules captured. FIG. 8A—Synthetic transcripts from pGEM constructs designed with 30 (lane 1), 149 (lane 2 and 3) A's were reverse transcribed using the Universal Reverse primer to which the T7 promoter sequence was added, then 2 μL of each RT reaction was used as template in 30-cycle PCR using forward and reverse primers containing T7 promoters. Lanes 4 and 5 are negative controls (no template added to RT or PCR). 10 μL of each PCR was run on 2% agarose gel containing ethidium bromide. FIG. 8B—2 μL of PCRs shown in A was used as template for in vitro transcription with T7 RNA polymerase (lanes 1-4); lane 5 is a positive control plasmid with T7 promoter. 2 μL of each 20 μL reaction were run as for A. For FIGS. 8A and 8B, mw was pUC19/Sau3A restriction fragments of 955, 585, 342, and 258 bp. FIG. 8C—34, of transcription reactions 1-4, was added to various amounts of streptaviden-agarose beads that had been coupled to a 5′ biotinylated oligo containing the T7 promoter sequence, incubated 30 min. at room temp, and then the beads were recovered by centrifugation, washed once in PBS, then suspended in 100 μL of PBS containing 1:10,000 dilution of SYBR Green 1. Tubes with minimal signal (14 and 15) were negative controls, either using beads not coupled to the T7 capture oligo or using transcription reactions made from negative-control WT rxn 4. Tubes showing SYBR signal (11, 12 and 13) contained positive WT reactions 1-3 added to different amounts of T7 oligo-coupled beads.

### PROPHETIC EXAMPLE 1

Development of the ALL-Tail for next generation sequencing. A library of forward primers for amplification of a panel of 100 different mRNAs or a random sequence to enable genomic scale studies may be prepared. The forward primers for individual mRNAs will be determined by bioinformatics analysis. Gene specific primers will be identified that meet the following two criteria: (a) are within 200 bases of the consensus starting position of the poly(A) tail; (b) have similar Tm's (within 2 deg) that are compatible with the Tm's of the primer that hybridizes to the 3′ linkers. The random forward primer will be designed to meet 3 main criteria: (a) lack runs of A's or T's at the 3′ end; (b) lack any complimentary to the linker sequences; (c) have similar Tm's (within 2 deg) that are compatible with the Tm's of the primer that hybridizes to the 3′ linkers. We routinely used linker-complementary reverse primers with Tm's of ˜47° C. during development of our assay, but we have designed and successfully used other adenylated linkers and associated reverse primers with varying Tm's.

### PROPHETIC EXAMPLE 2

The use of the ALL-TAIL assay kit as described in FIG. 6, can be used to study circadian rhythm and light/dark cycle genes including but not limited to Arnt1, Bmal1, Mop3, Per1-3 and Cry1. Our data demonstrates poly(A) tail length variability of Arnt1 genes during light and dark cycles. The ALL-TAIL assay kit can be used to identify genes with endogenous circadian rhythm from non light/dark cycle genes.

### PROPHETIC EXAMPLE 3

The use of the ALL-TAIL assay kit as a cancer diagnostic tool. FIG. 7 demonstrates poly(A) tail length of an oncogene: c-fos from two tissues of similar origin, one a cancer cell line (A549) and one a non-tumorigenic cell line (WI38) had different poly(A) tail lengths. Using the ALL-TAIL assay to identify poly(A) tail lengths of several oncogenes, 3′UTR sequence/length could demonstrate usefulness as a prognostic and or diagnostic cancer tool. Longer or shorter poly(A) tail lengths could be a global indicator of the status and or progression of a particular cancer. Length or sequence variation of 3′UTRs of oncogenes could also be indicative of cancer type, progression and stage.

### PROPHETIC EXAMPLE 4

Linker ligation approach for detecting miRNA molecules using end point or real time PCR. In this case, the linker is ligated to total or enriched small RNA preparations. The RNA is reverse transcribed using a primer complementary to the linker. Subsequent PCR is performed using a primer specific to the linker and an upstream primer complementary to the miRNA. The upstream primer may be fully complimentary to the miRNA or in some cases contain an overhand. Detection for real time PCR can be any of the methods known in the art.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following uses.

1. A method of determining the sequence of an RNA molecule, comprising:
ligating an oligonucleotide sequence to the 3′ end of the RNA molecule;
reverse transcribing the ligated RNA molecule using a primer complementary to all or part of the ligated oligonucleotide sequence;
amplifying the transcribed RNA to produce a mixture comprising multiple copies of the RNA molecule; and
determining the sequence of the RNA molecules in the mixture.

2. The method of claim 1, wherein the RNA molecule has a poly(A) tail, and wherein determining the sequence of the RNA molecule comprises determining the poly(A) tail length of the RNA molecule.

3. The method of claim 1, wherein the oligonucleotide sequence comprises an ATP moiety coupled to the 5′ end, wherein the ATP moiety is removed during ligation of the oligonucleotide sequence to the 3′ ends of the RNA molecule.

4. The method of claim 3, wherein the ligation of the oligonucleotide sequence to the 3′ end of the RNA molecule is performed in the absence of added ATP during ligation.

5. The method of claim 1, wherein the oligonucleotide sequence comprises a dideoxy nucleotide coupled to the 3′ end, wherein the ligated RNA molecule comprises the dideoxy nucleotide coupled to the 3′ end of the RNA molecule.

6. The method of claim 1, wherein the oligonucleotide sequence has a melting temperature between about 40° C. to about 60° C.

7. The method of claim 1, wherein amplifying the transcribed RNA comprises using a Forward primer that hybridizes to a sequence near the 3′ end of the unique sequence of the RNA molecule and a Reverse primer that hybridizes to a sequence within the ligated oligonucleotide sequence.

8. The method of claim 1, wherein the determining the sequence of the RNA molecules comprises mixing the multiple copies of the RNA molecule with a dye, wherein the signal strength of the bound dye corresponds to the length of the RNA molecule.

9. The method of claim 1, wherein ligating an oligonucleotide sequence to the 3′ end of the RNA molecule is performed in the presence of a bridging oligonucleotide.

10. The method of claim 1, wherein determining the sequence of the RNA molecules in the mixture comprises determining 3′ end modifications of mRNAs from all biological organisms and systems.

11. The method of claim 1, wherein determining the sequence of the RNA molecules in the mixture comprises assessing the 3′ end proximal sequences of RNA precursors of all types from all biological organisms and systems.

12. The method of claim 1, wherein determining the sequence of the RNA molecules in the mixture comprises determining sites of transcription termination from all biological organisms and systems.

13. The method of claim 1, wherein determining the sequence of the RNA molecules in the mixture comprises analyzing 3′ proximal sequences of RNA processing and decay intermediates from RNAs of all types from all biological organisms and systems.

14. The method of claim 1, wherein determining the sequence of the RNA molecules in the mixture comprises determining and analyzing the 3′ proximal sequences of intergenic transcription products from all biological organisms and systems.

15. The method of claim 1, wherein the RNA molecule is an RNA molecule produce under disease state conditions, wherein the method further comprises determining the difference of poly adenylated tail length between RNA molecules in the disease state and RNA molecules in a non-disease state.

16. A method for comparing 3′UTR/polyA sequences between RNA molecules, comprising: ligating an oligonucleotide sequence to the 3′ ends of an RNA molecule;
reverse transcribing the ligated RNA molecule using a primer complementary to all or part of the ligated sequence;
amplifying a specific 3′UTR/polyA sequence using a Forward primer that hybridizes to a sequence near the 3′ end of the unique sequence of the RNA molecule and a Reverse primer that hybridizes to a sequence within the ligated oligonucleotide sequence,
mixing a portion of the amplified product with T7 RNA polymerase, rNTPs, and an appropriate buffer containing one or more divalent cations to form an amplification mixture;
incubating the amplification mixture to allow transcription of both strands of the PCR amplicon to produce multiple RNA copies of each strand of the amplicon, wherein the strands will hybridize to create double-stranded RNA with single-stranded tails complementary to the first 16 bases of the T7 promoter;
adding a portion of the incubated amplification mixture to a matrix to form a matrix mixture, wherein the matrix is coupled to oligonucleotide having a sequence comprised of at least the first 16 bases of the T7 promoter;
incubating the matrix mixture to allow hybridization of the single-strand tails complementary to the T7 promoter, generated during incubation of the amplification mixture, with the oligonucleotide on the matrix;
recovering the matrix;
resuspending the matrix with associated double-strand RNA in a suitable buffer containing suitable double-strand nucleic acid-specific dye; and
assessing the extent of staining of the material treated with the double-strand nucleic acid-specific dye, wherein the extent of staining is positively correlated with the sequence of the RNA molecule.

17-34. (canceled)
